(19) United States
(12) Patent Application Publication
(10) Pub. No.: US 2024/0342210 A1
(43) Pub. Date: Oct. 17, 2024

(54) COMPOSITIONS AND METHODS FOR TREATMENT OF OBSTRUCTIVE SLEEP APNEA

(71) Applicant: The General Hospital Corporation, Boston, MA (US)

(72) Inventors: Lilit Garibyan, Newton, MA (US); William A. Farinelli, Boston, MA (US); Richard Rox Anderson, Boston, MA (US)

(73) Assignee: The General Hospital Corporation, Boston, MA (US)

(21) Appl. No.: 18/701,054

(22) PCT Filed: Oct. 14, 2022
(86) PCT No.: PCT/US2022/046663

§ 371 Date: Apr. 12, 2024

Related U.S. Application Data
(60) Provisional application No. 63/255,733, filed on Oct. 14, 2021.

(51) Int. Cl. A61K 33/00; A61K 9/00; A61K 47/10; A61P 11/00

(52) U.S. Cl. CPC A61K 33/00; A61K 9/0019; A61K 47/10; A61P 11/00

(57) ABSTRACT

Disclosed herein are methods that allow for treating sleep apnea by removing adipose tissue and increasing collagen production in a patient's upper airways by injecting ice slurry comprising ingredients such as sterile water, a biocompatible surfactant such as glycerol, and saline into the target area.

### BACKGROUND OF THE INVENTION

Obstructive sleep apnea (OSA) is chronic disease which results in excessive daytime sleepiness and cognitive impairment leading to an increased risk of motor vehicle accidents. However, the exact pathophysiology of OSA is not yet clear. There is accordingly an unmet need for targeted therapy minimally invasive, and painless method for treatment in this patient population. Here, we disclose a method for reducing upper airway fat and inducing new collagen formation in the tongue by cryolipolysis as a novel method of OSA treatment.

Obesity is the most important risk factor for obstructive sleep apnea (OSA). Obesity is thought to increase the size of soft-tissue structures in the upper airway by various mechanisms, one of which is direct deposition of fat within these tissues. Increased upper airway adipose tissue, specifically deposited in the lateral parapharyngeal fat pads, the uvula, and the tongue is thought to play a major role in the pathogenesis of sleep apnea. Volume of pharyngeal walls, tongue and total soft tissue is reported to be larger in subjects with sleep apnea.

Although the amount of visceral fat in the neck is thought to be involved in the enlargement of the upper airway soft tissue structures, it may not be the most important factor. Other factors besides obesity (in addition to age, sex, craniofacial size, and ethnicity) may be important in mediating the increase in the size of the tongue and lateral pharyngeal walls. Reduced tongue stiffness has been shown in patients with OSA compared to—age and—BMI matched control. This leads to increased tongue laxity and collapsibility seen in patients with OSA, suggesting there are fundamental differences in the mechanical properties of the tongue tissue in patients with OSA, and these changes are not the result of only age or obesity. The invention disclosed herein includes a new method to induce new collagen formation in the tongue to decrease tongue laxity and improve obstructive sleep apnea irrespective of fat loss.

### BRIEF DESCRIPTION OF FIGURES

The patent or application file contains at least one drawing originally in color.

FIG. 1 shows the code for the collagen content score.

FIGS. 2A-B show tissue temperature and histology images of the base of the tongue for pig #4 following a sequence of ice slurry injections. FIG. 2A shows tissue temperature recorded with a thermocouple embedded in the needle administering the ice slurry injection. Pig #4 was injected sequentially 3 times with 20 mL of ice slurry every time the tissue temperature rose back to zero. FIG. 2B shows histology images from biopsies of the base of the tongue at 2 months post ice slurry treatment. Blue is trichrome stain which highlights new collagen formation in places where the fat was lost. FIG. 2C shows representative ultrasound images of the injection site in the base of the tongue before (left) and after (right) injection of slurry. The yellow arrow shows the hyoid bone, and the blue arrow marks the base of the tongue. The red arrow shows the tip of the injection needle, and the white arrow marks the injected slurry at the base of the tongue.

FIG. 3 A-B show tissue temperature and histology images of the base of the tongue for pig #5 following a sequence of ice slurry injections. FIG. 3A shows tissue temperature recorded with thermocouple embedded in the needle. The pig was injected sequentially 3 times with 20 ml, 17 ml, and 20 ml of ice slurry, respectively, every time the tissue temperature rises back to zero. FIG. 3B shows histology images from biopsies of the base of the tongue at 2 months post ice slurry treatment. The blue in FIG. 3B highlights new collagen formation.

FIG. 4 show tissue temperature and histology images of the base of the tongue for pig #6 following a sequence of ice slurry injections. FIG. 4A shows tissue temperature recorded with thermocouple embedded in the needle. The pig was injected sequentially 6 times with 17 ml, 12 ml, 5 ml, 14 ml, 5 ml, and 7 ml of ice slurry, respectively, every time the tissue temperature rises back to zero. FIG. 4B shows histology images from biopsies of the base of the tongue at 2 months post ice slurry. The blue in FIG. 4B highlights new collagen formation.

FIG. 5 shows collagen area measurements and damage score following ice slurry injection in pigs #4, #5, and #6.

FIG. 6 shows the safety of injection of the ice slurry at the base of the tongue. FIG. 6A shows the body weight of animals in the ice-slurry group and the RT control group at baseline and 8 weeks post injection. FIG. 6B shows a representative gross image of the tongue with biopsy sites at the base of the tongue marked in red. FIG. 6C includes representative histological images of the base of the tongue at untreated sites, sites injected with room temperature (RT) control solution, and sites injected with ice slurry. The H&E sections (top raw) show adipose tissue (the white sections) admixed with muscle fibers (the red sections) with a similar ratio between the untreated tongue tissue and the RT control tissue. The treated panel shows adipose tissue reduction and increased fibrosis (pink wavy areas in between darker muscle fibers within adipose tissue). The trichrome sections (second row from the top) show newly formed collagen stained in light blue. The treated panel (right-most column) shows abundant new collagen deposition. The Luxol blue sections (third row from the top) show myelinated fibers stained in blue. The treated panel (right-most column) shows similar levels of myelinated fibers compared to the untreated and RT control sections, demonstrating that the myelinated fibers were not altered by the slurry injection. The Neurofilament immunostaining sections (fourth row from the top) show axons of neurons in brown. Nerve axons were preserved in all treatment groups. The scale bar on these images is 100 μm.

FIG. 7 A-B show histological images of neck fat pads of a mouse and adjacent salivary glands after injection of ice slurry or room temperature control solution in the anterior neck fat pad. FIG. 7A shows representative histological images of anterior neck fat pads and adjacent salivary glands 12 weeks after treatment with slurry or room temperature control solution. The scale bars on FIG. 7A are 10 μm. FIG. 7B shows the body weight of mice in slurry-treated and control mice at baseline and at 12 weeks post injection.

FIG. 8A-B show Axial MRI imaging of the anterior neck fat pad post injection of ice slurry or post injection of room temperature control solution. FIG. 8A shows Axial MRI imaging with outlined region of interest (red) and changes in fat volume (blue); the scale bar is 1 cm. FIG. 8B shows the fractional anterior neck fat volume in slurry-treated and control mice at baseline and at 12 weeks post injection. FIG. 8C shows changes in the fractional anterior neck fat volume per animal weight in slurry-treated and control mice. Age-matched male New Zealand mice were used in this study; n=13-14 per group.

FIG. 9 shows changes in fractional anterior neck fat pad volume of mice from baseline per body weight between ice slurry treatment group and RT control group.

FIG. 10 shows an anterior transcervical injection approach and route.

### DEFINITIONS

The following are definitions of terms used in the present specification. The initial definition provided for a group or term herein applies to that group or term throughout the present specification individually or as part of another group, unless otherwise indicated. Unless otherwise defined, all technical and scientific terms used herein have the same meaning as commonly understood by one of ordinary skill in the art.

The singular forms “a”, “an” and “the” include plural reference unless the context clearly dictates otherwise. The use of the word “a” or “an” when used in conjunction with the term “comprising” in the claims and/or the specification may mean “one,” but it is also consistent with the meaning of “one or more,” “at least one,” and “one or more than one.”

As used herein the term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent up or down (higher or lower).

As used herein, the term “subject” refers to a vertebrate animal. In one embodiment, the subject is a mammal or a mammalian species. In one embodiment, the subject is a human. In one embodiment, the subject is a healthy human adult. In other embodiments, the subject is a non-human vertebrate animal, including, without limitation, non-human primates, laboratory animals, livestock, racehorses, domesticated animals, and non-domesticated animals. In one embodiment, the term “human subjects” means a population of healthy human adults.

As used herein, the term “patient” refers to a human or animal.

The term “mammal” includes, but is not limited to, a human, mouse, rat, guinea pig, dog, cat, horse, cow, pig, or non-human primate, such as a monkey, chimpanzee, baboon, or rhesus. In one embodiment, the mammal is a human.

### DETAILED DESCRIPTION OF THE INVENTION

There is presently no injectable device that can decrease upper airway fat or increase new collagen formation that is completely physiologic and non-toxic to surrounding tissue. The present disclosure provides methods that are physiologic and biocompatible, as the compositions used therein comprise ingredients such as sterile water, a biocompatible surfactant such as glycerol, and saline. Moreover, the compositions used in the methods disclosed herein are injectable, thus providing for compositions that infiltrate the target area. Therefore, exact precision as to the location of injection is not required, and instead injection in the vicinity of the target area is efficacious. In addition, due to the injectable and infiltrating nature of the slurry, it takes minutes to complete this procedure as compared to the hours it takes for other tongue debulking procedures, such as applying topical cooling to the tongue's surface or surgically removing tongue tissue (which also requires the use of general anesthesia). The disclosed methods therefore enable treatment of obstructive sleep apnea in a physician's office in a matter of minutes.

The methods disclosed herein do not damage surrounding tissue and are adipose tissue selective. The methods disclosed herein have an injection sequence and tissue cooling duration that lead to increasing collagen. Further the method disclosed herein does not induce scaring or any damage to important surrounding tissue. Therefore, this disclosure provides a method for treating sleep apnea by removing adipose tissue and increasing collagen production in a patient's upper airways by injecting ice slurry into the target area.

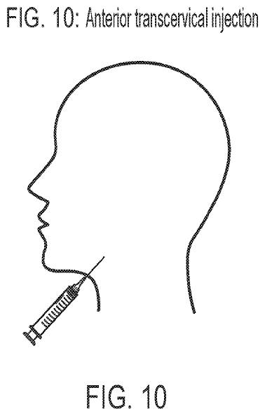

The methods and disclosure herein provide many unexpected results. In one embodiment, the disclosed ice slurry can be injected from an anterior transcervical approach (as shown in FIG. 10). This is advantageous because it allows easy access to adipose tongue tissue as the injection target is located about 2-4 cm from the site where the needle is inserted into a patient's neck. In another embodiment, the ice slurry can be injected via another route, such as from the top surface of the tongue. In another embodiment, ultrasound can be used to identify the patient's anatomy and the location of injection. Ultrasound also enables visualization of the location of the ice sphere after injection. Ultrasound guidance may increase the safety of such injections.

In one embodiment, the methods disclosed herein provide an advantage of allowing up to 60 mL of ice slurry to be injected to the target site. In a preferred embodiment, 60 mL of ice slurry is injected in a sequence of 5, 10, or 20 mL amounts. The structure of the tissue at the base of the tongue, which is a mixture of fat and muscle tissue, allows the slurry to infiltrate and spread in the tissue. This is in contrast to injecting slurry into other, denser tissues where an ice ball may form at the site of injection. The amount of slurry that can be injected allows a dispersive treatment across the base of the tongue.

In one embodiment, the methods disclosed herein provide an advantage of not damaging other tissue in the tongue. In a preferred embodiment, the methods disclosed herein do not damage nerves that are located in the tongue. This is due in part to injecting the ice slurry in the middle of the tongue. In one embodiment, the methods disclosed herein provide an advantage of having an easy route of injection that lessens patient discomfort. By accessing the injection site through a patient's neck, a physician is able to easily and quickly inject ice slurry without causing a patient to gag or

### Non-Limiting Embodiments of the Subject Matter

Embodiments of the invention provide an injectable ice slurry that can be used to treat obstructive sleep apnea, by reducing upper airways adipose tissue and/or by increasing collagen formation in the tongue. Such slurries can target and disrupt desired tissue through the heat extracted from adjacent tissue during melting of the ice component of the slurry.

In one non-limiting embodiment the ice slurry is injected around the upper airway that is targeted for treatment of obstructive sleep apnea. In one embodiment, injecting ice slurry around the upper airway reduces upper airways adipose tissue. In one embodiment, the slurry is injected through a needle that is inserted via an anterior transcervical approach. In one embodiment, the slurry is injected without piercing any surface of the tongue. In one embodiment, the slurry is injected through a needle that is inserted into a patient's tongue from underneath the patient's chin.

Enlargement of fat surrounding the upper airway is a newly proposed factor for upper airway obstruction in obstructive sleep apnea (OSA), particularly in obese patients. OSA causes significant morbidity and mortality. It has been reported that fat can be selectively targeted and removed by tissue cooling, which led to a popular non-invasive alternative to liposuction. Recently, we developed biocompatible, injectable ice slurry composed of normal saline and glycerol, as a novel method to remove subcutaneous fat. Fat deposition in the neck results in increased neck circumference which shows a strong correlation with apnea severity in patients. New Zealand Obese (NZO) mice have increased volume of neck fat around upper airway and are used as translational model for OSA. This study demonstrated safety and efficacy of an injectable ice slurry in selective destruction of fat deposits in the neck in NZO mice.

In certain aspects, the sterile ice slurry reduces upper airway adipose tissue. In an embodiment, the ice slurry includes sterile water and ice particles. In one embodiment, the ice particles in the sterile ice slurry have a largest cross-sectional diameter of less than 2 millimeters.

The sterile ice slurry is cooled to a predetermined temperature. In one embodiment, the predetermined temperature is between minus 10° C. and 0° C. In one embodiment, the desired tissue region includes the upper airway adipose tissue. In one embodiment, the upper airway adipose tissue is the anterior neck fat pad. In one embodiment, injection of the sterile ice slurry reduces the volume of the anterior neck fat pad.

In another embodiment, the upper airway adipose tissue is tongue fat. In a further embodiment, the tongue fat is the base of tongue adipose tissue. In one embodiment, the injectable slurry includes a plurality of sterile ice particles and one or more freezing point depressants. The freezing point depressants can also alter the viscosity of the slurry, prevent agglomeration of the ice particles, increase thermal conductivity of fluid phase, and otherwise improve the performance of the slurry. In one embodiments injection of the sterile ice slurry reduces the volume of the tongue fat.

In order to ensure that the slurry can be injected through into a subject through a needle or a catheter, the size of the ice particles can be controlled. A slurry will be injectable if all or most (e.g., greater than about 50% by quantity, greater than about 75% by quantity, greater than about 80% by quantity, greater than about 90%, by quantity, greater than about 95% by quantity, greater than about 99% by quantity, and the like) of the ice particles have a largest cross-sectional dimension (i.e., the largest distance between any two points on the surface of the ice particle) no greater than half of the internal diameter of the vessels (e.g., needles, cannulae, catheters, tubing, and the like) to be used. For example, if the slurry is to be injected using a catheter having a 3 mm internal diameter, the ice particles will preferably have a largest cross-sectional dimension less than or equal to 5 about 1.5 mm. In some embodiments, the ice particles have a mean largest cross-sectional dimension of 1 mm or less.

One or more freezing point depressants can be mixed with the ice to form sub-0° C. slurries that remain injectable. Suitable freezing point depressants include biocompatible compounds such salts (e.g., sodium chloride), Lactated Ringer's solution, glucose, biocompatible surfactants such as glycerol (also known as glycerin or glyceline), other polyols, other sugar alcohols, and/or urea, and the like. In particular, biocompatible surfactants such as glycerol is believed to cause the ice particles to shrink and become rounder and also serves as a cryoprotectant for non-lipid-rich cells. Other exemplary biocompatible surfactants include sorbitan esters of fatty acids, polyoxyethylene sorbitan monooleate (also known as polysorbate 80 and available under the TWEEN® 80 trademark from Croda Amelicas LLC of New Castle, Delaware), sorbitan monooleate polyoxyethylene sorbitan monolaurate (also known as polysorbate 80 and available under the TWEEN® 80 trademark from Croda Americas LLC of New Castle, Delaware), lecithin, polyoxyethylene-polyoxypropylene copolymers (available under the PLURONICS® trademark from BASF Corporation of Mount Olive, New Jersey), sorbitan trioleate (available under the SPAN® 85 trademark from Sigma-Aldrich of St. Louis, Missouri) and the like. Injectable slurries can be configured to have a desired temperature and to extract a desired amount of heat per unit of volume or mass of slurry. Specifically, the solute (i.e., freezing point depressant) concentration dictates the temperature of the slurry and the ice content of the slurry determines the amount of heat extracted by the slurry.

For selectively-destructive slurries that target the relative vulnerability of lipid-rich cells, the slurry is preferably isotonic relative to the subject's cells. For example, slurries including normal saline and 20% glycerol were able to target lipid rich cells while avoiding acute unselective necrosis. Broadly destructive slurries can achieve colder temperatures and greater destructive power by increasing the solute concentration (e.g., to 20% w/v saline) to form a hypertonic solution that will also disrupt cells through osmotic pressure. As the ice melts, the solute concentration will decrease.

The injectable slurries can contain varying proportions of ice. For example, the slurries can contain between about 10% and about 5% ice by weight, between about 10% and about 20% ice by weight, between about 20% and about 30% ice by weight, between about 30% and about 40% ice by weight, between about 40% and about 50% ice by weight, between about 50% and about 60% ice by weight, between about 60% and about 70% ice by weight, and greater than about 50% ice by weight. (The proportions by volume will be similar due to the densities of solid and liquid water.)

### Methods of Preparing Slurries

Slurries can be prepared using a variety of methods. Any known method of creating ice slurry is contemplated here.

In one embodiment, a slurry is prepared using a commercially-available ice slurry generator such as those available under the MODUPAK™ DEEPCHILL™ trademark from Sunwell Technologies Inc. of Woodbridge, Ontario. Commercially-available slurry generators include scraped surface generators that wipe away (e.g., with blades, augers, brushes) small ice crystals from a chilled surface and mixed with water, direct contact generators in which an immiscible primary refrigerant evaporates to supersaturate the water and form small smooth crystals, and super cooling generators in which water is supercooled and released through a nozzle into a storage tank.

### Slurry Storage and Further Processing

Both the slurries described herein and precursor ice particles can be stable for years if held below the freezing point of the solution or the ice particles. In order to guard against growth or agglomeration of ice crystals, it is preferable to store the slurry at a temperature below the freezing point and then let the slurry reheat to the desired injection temperature.

Either method described above can be performed by a single actor at a single location at a single time or can be performed by one or more actors at one or more locations at one or more times. For example, small stable ice particles can be packaged and shipped using standard cold shipping methods and stored in a standard freezer (e.g., at −20° C.). The ice particles can be combined with one or more additional additives in the clinic shortly or immediately prior to injection. As discussed herein in greater detail, the additives can, for example, be biocompatible solutions, contain a biocompatible surfactant such as glycerol, and be precooled (e.g., to a temperature approximating the desired temperature of the slurry at the time of injection).

### Slurry Delivery

The injectable slurry can be introduced using various parenteral delivery systems and techniques including gravity flow, injection through a syringe, a cannula, a catheter, tubing, and/or a pump, and the like. A control device can control the flow rate, volume, and or pressure of the injected slurry in order to extract a desired amount of heat from tissue adjacent to the injection site.

Optionally, an imaging technique such as ultrasound, magnetic resonance, x-ray, and the like can be utilized to verify the proper positioning of the injection device and/or the slurry. In particular, ice is a very strong reflector of ultrasound, while lipid rich cells are poor reflectors of ultrasound.

This method can be repeated one or more times for the same injection site and/or target tissue. Multiple injections can be performed in serial, overlapping, or parallel manner.

In one embodiment the sterile ice slurry is delivered to the desired tissue region via a pump, and melted slurry is suctioning from the desired tissue region. In another embodiment, the ice slurry is injected to the desired target region from a syringe and through a needle.

In certain aspects, the sterile ice slurry increases new collagen formation in the tongue. In other certain aspects, the sterile ice slurry decreases the amount of adipose tissue present in the tongue. In one embodiment, the ice slurry includes water and ice particles. In one embodiment, the ice particles in the sterile ice slurry have a largest cross-sectional diameter of less than 2 millimeters.

The sterile ice slurry is cooled to a predetermined temperature. In one embodiment the predetermined temperature is between minus 10 degrees celcius and 0 degrees celcius. In one embodiments the desired tissue region includes the tongue. In one embodiment, the injectable slurry includes a plurality of sterile ice particles and one or more freezing point depressants. The freezing point depressants can also alter the viscosity of the slurry, prevent agglomeration of the ice particles, increase thermal conductivity of fluid phase, and otherwise improve the performance of the slurry.

In one embodiment, the ice slurry injection is repeated one or more times for the same injection site and/or target tissue. In a preferred embodiment, the injection is repeated 1-6 times for the same injection site and/or target tissue. In one embodiment, the volume of ice slurry for each injection is between 5 ml and 50 ml. In one embodiment, the injection of the sterile ice slurry stiffens the tongue by increasing collagen production. The injection of the sterile ice slurry may also reduce adipose tissue in the tongue.

### EXAMPLES

In order that the invention described herein may be more fully understood, the following examples are set forth. The synthetic examples described in this application are offered to illustrate the compounds and methods provided herein and are not to be construed in any way as limiting their scope.

### Example 1—Ice Slurry Induced New Collagen Formation with Biopsy Sites on the Tongue and Histologic Grading by Pathologist

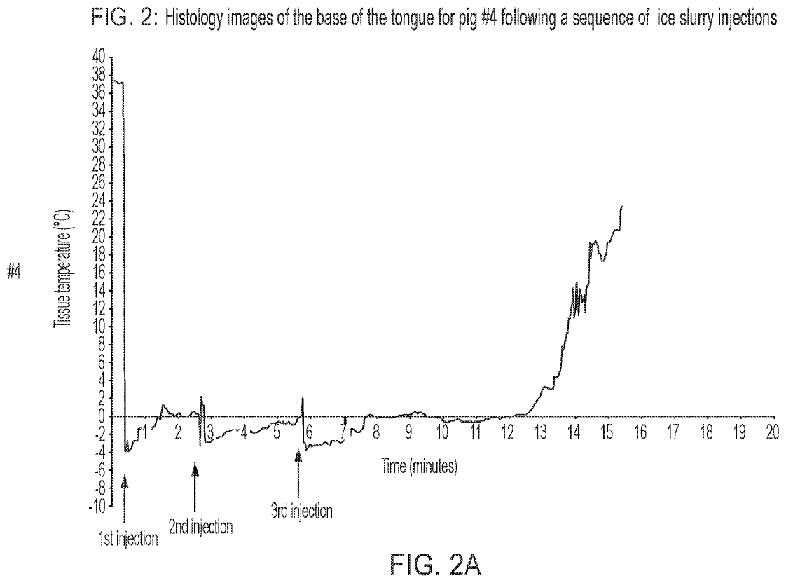

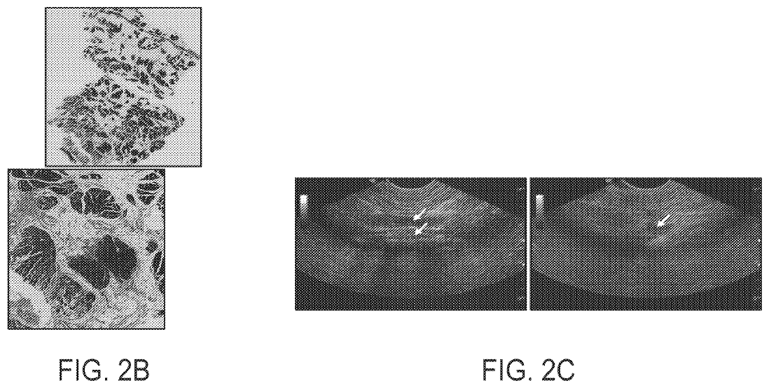

The animal study was approved by Massachusetts General Hospital (MGH) IACUC and animals were housed at MGH in accordance with animal care regulations. A total of 6 Yorkshire female swine 4-6 months of age were used in this study. Four Yorkshire swine in the test group were injected with ice-slurry (−6° C.) and two control swine were injected with room temperature “slurry solution” in the base of the tongue. Slurry composed of normal saline (0.9% sodium chloride) and 10% glycerol was used as previously described. Under general anesthesia, ultrasound (US) guidance was used to pass a 14 g needle through the anterior neck into the base of tongue. Anatomic landmarks on the pig model included the posterior mandible and the hyoid bone. The needle was inserted just superior to the hyoid and guided into the left or right base of tongue noting tissue displacement when the needle was passed perpendicular to the probe. A laryngoscope and a rigid 0-degree laryngeal telescope were used to confirm placement of the needle in the base of tongue. A total of 60 ml ice slurry or RT control was injected in consecutive injections. Tissue temperature at the site of injection was recorded using a thermocouple (FIG. 2C). Animals were observed clinically for 2 months after the injection for potential adverse effects. At 2 months post treatment, animals were sacrificed, and tongue tissue was harvested for gross and histologic analysis. Multiple biopsy specimens were collected from the base of the tongue. Biopsies were fixed in 10% formalin, embedded in paraffin, sectioned at 5 μm, then stained with hematoxylin and eosin (H&E). Trichrome staining was used to examine collagen deposition. Immunohistochemistry for neurofilament and special Luxol blue staining were performed to study axons and myelin structure in nerves. A board-certified pathologist blindly assessed the biopsy samples to evaluate the histologic changes in adipose tissue, muscles, nerves, vessels, salivary glands, lymphoid tissue, and epithelium of the tongue.

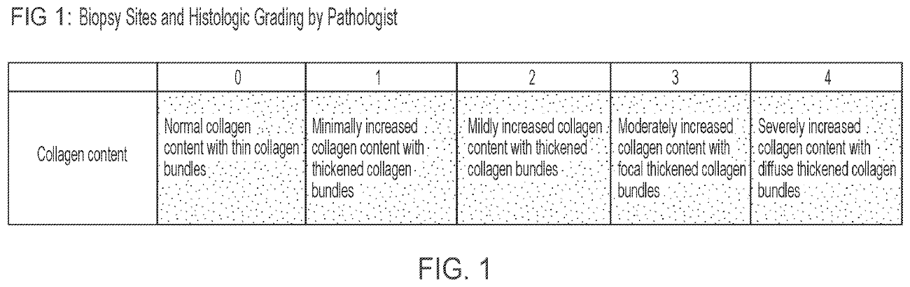

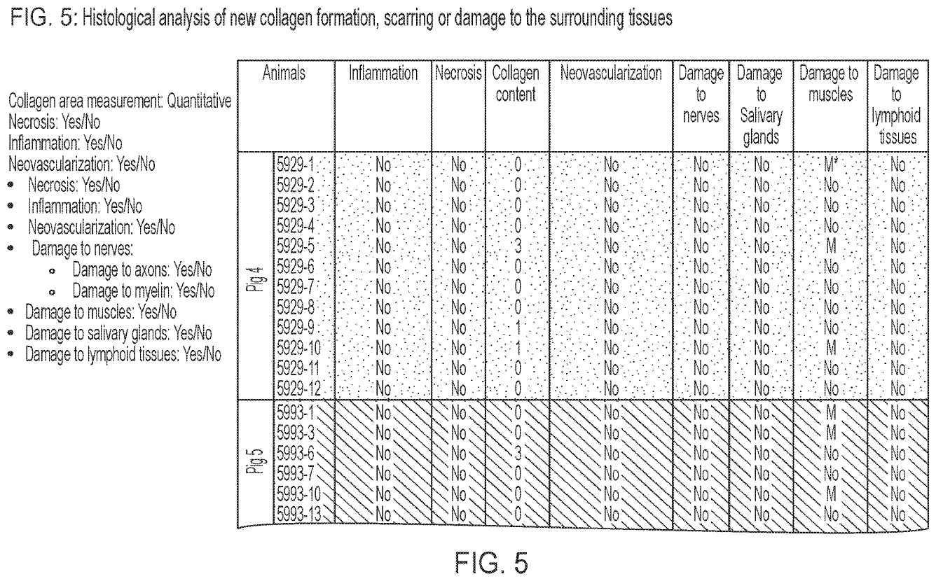

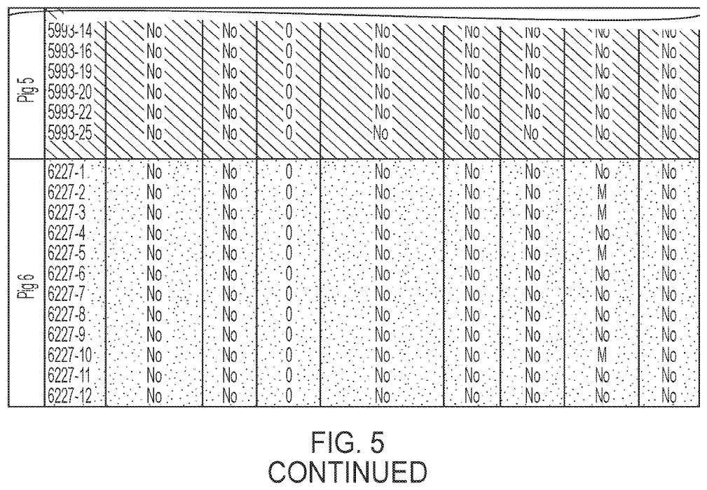

Experiments in four pigs demonstrate that injection of ice slurry is safe and changes the collagen content in the tongue as shown by examination of gross and histological imaging of the tongue; and the collagen content score on the treated side of the tongue compared to the untreated side (see FIG. 1-6). This experiment demonstrated the increase in collagen content in the tongue after ice slurry administration.

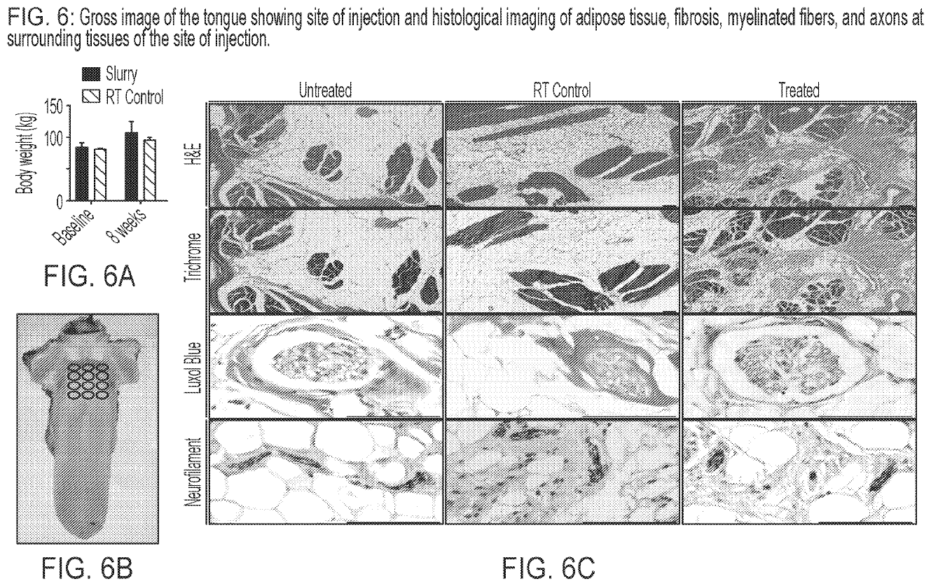

Ice slurry treatment was provided as follows: A total of 60 ml of ice slurry at around −6° C. (0.9% sodium chloride with 10% glycerol) or 60 ml of room temperature slurry ((0.9% sodium chloride with 10% glycerol) was injected in the tongue of pigs, under brief anesthesia with inhalational isoflurane (1 to 3% with 1 to 1.5 l/minute oxygen), using a syringe with a needle. The pigs received multiple, sequential injections of 5-20 ml of ice slurry or room temperature solution per injection. Subsequent injections were provided when the tissue temperature rose back to zero following the previous injection. A 14 or 15-gauge hypodermic needle was used for the injections. Twelve different sites of the tongue base were biopsied for histologic analysis (see FIG. 6B which shows the location of the biopsy). Semi-quantitative score was used by board certified and blinded pathologist to quantify the collagen content in all 12 biopsies from each pig (FIG. 1A).

Thermocouple recordings demonstrated that tissue temperature below 0° C. for several minutes was achieved (FIG. 2A), which is sufficient for obtaining selective fat loss. US imaging was used to confirm the location of injection needle and ice-ball formation in the base of the tongue (FIG. 2C).

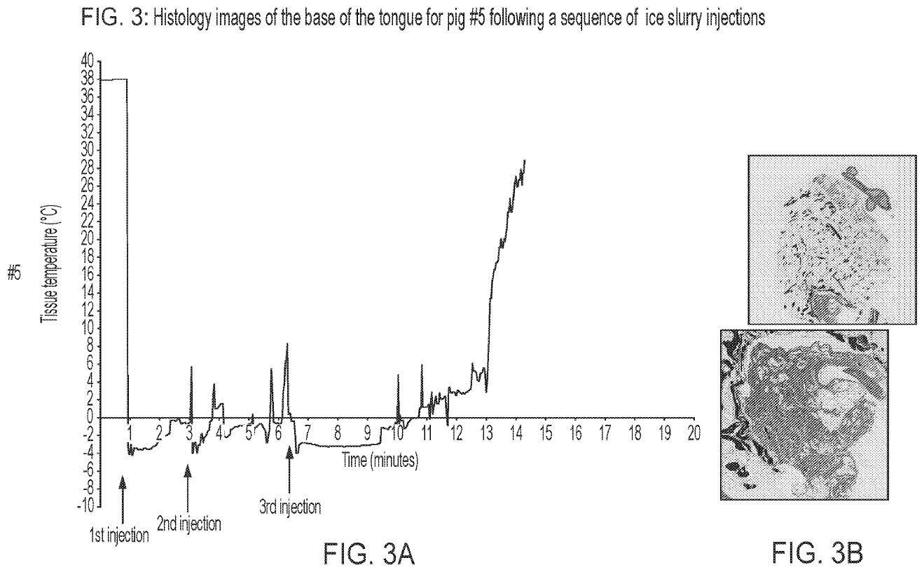

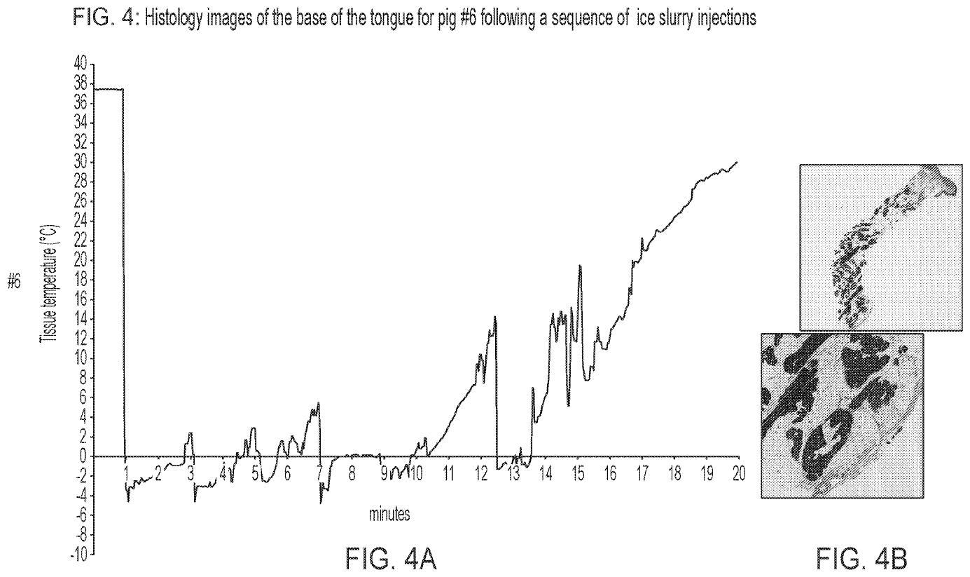

As shown in FIGS. 2A-B and 3A-B, at 2 months after injection of ice slurry in the tongue of pig #4 and #5, tissue temperature was recorded with thermocouple imbedded in the needle. As shown in FIGS. 2A-B, after 3 injections of 20 ml of ice slurry to the base of the tongue of pig #4, tissue temperature was recorded to test the rate of tissue cooling and the duration of time tissue temperature below 0 degrees Celsius. The histology samples collected from the tongue at 2 months post ice slurry treatment were stained with Trichrome to highlight collagen in blue and these samples were semi-quantitatively scored for new collagen formation. Thermocouple recordings demonstrated that tissue temperature below 0° C. for several minutes was achieved (FIG. 1A), which is sufficient for obtaining selective fat loss (7). US imaging was used to confirm the location of injection needle and ice-ball formation in the base of the tongue (FIG. 1B). As shown in FIGS. 3A-B, after 3 sequential injections of 20, 17, and 20 ml of ice slurry to the base of the tongue of pig #5, tissue temperature was recorded to test the rate of tissue cooling and the duration of time tissue temperature below 0 degrees Celsius. The histology samples collected from the tongue at 2 months post ice slurry treatment were stained with Trichrome to highlight collagen in blue and these samples were semi-quantitatively scored for new collagen formation. As shown in FIGS. 4A-B, after 6 sequential injections of 17, 12, 5, 14, 5, and 7 ml of ice slurry on one side of the tongue of pig #6, tissue temperature was recorded to test the rate of tissue cooling and the duration of time tissue temperature below 0 degrees Celsius. The histology samples collected from the tongue at 2 months post ice slurry treatment were stained with Trichrome to highlight collagen in blue and these samples were semi-quantitatively scored for new collagen formation.

The data presented in FIGS. 2-4 show quantitatively by graphing the thermocouple recording that the tongue tissue of pig #4 and #5 injected with ice slurry was cooled to below 0 degrees Celsius after every injection and that the duration the tissue temperature was below 0 degrees Celsius extended after each injection (FIG. 2A, 3A, 4A). Pig #4 and pig #5 had the longest duration the tissue temperature was below 0 degrees Celsius, while pig #6 had the shortest duration of tissue temperature below 0 degrees Celsius. The data presented in FIGS. 2-5 also show, by semi-quantitative histologic grading, new collagen formation with ice slurry injections as indicated by blue highlights (FIG. 2B, 3B, 4B, 5). The blue in Trichrome stain indicated collagen. Pig #4 and pig #5 had displayed the most collagen formation, while pig #6 had very little to almost no new collagen formation indicating that cooling temperature and duration are important. The location of the collagen formation was at the base of the tongue which is where the injections were done, indicating that the collagen formation is a result of slurry injection and not a non-specific response.

Injection of ice-slurry to the base of the tongue was performed without complications. There was no evidence of tongue necrosis or airway obstruction after injection in either treatment or control arms. Animals maintained normal weight gain post treatment suggesting that feeding requiring the use of tongue was not altered (FIG. 6A). Examination of gross tongue tissue specimens at 2 months post-treatment showed any scarring or trauma (FIG. 6B). Histological analysis from the base of the tongue did not show damage to the surrounding muscles, nerves, or vessels. Immunohistochemistry for neurofilament and Luxol blue special stain showed normal nerve structure, axons, and myelin. Histologic evidence of selective fat loss along with new collagen deposition was observed in tongue tissue (FIG. 6C). No sign of inflammation, scarring or necrosis was observed.

### Example 2—Ice Slurry Induced Anterior Neck Fat Pad Size Changes

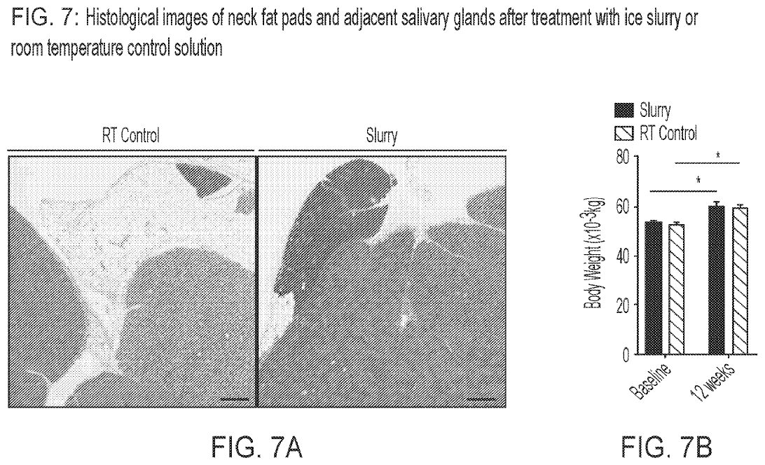

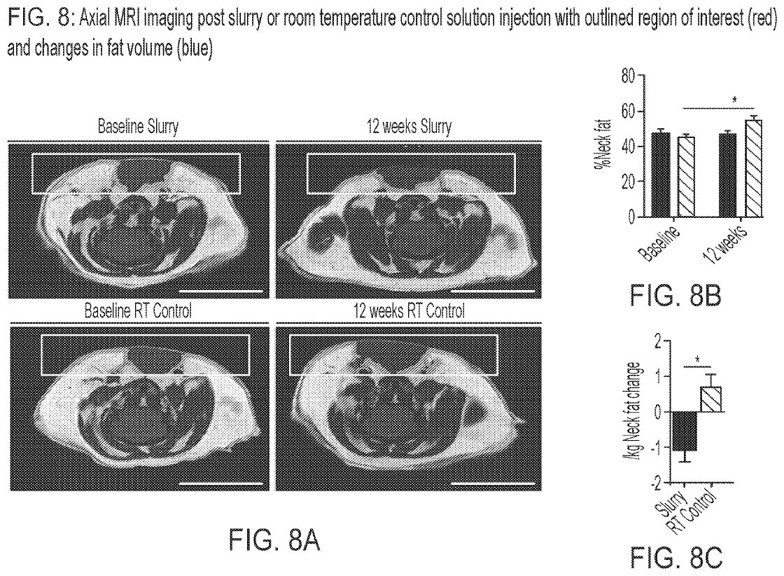

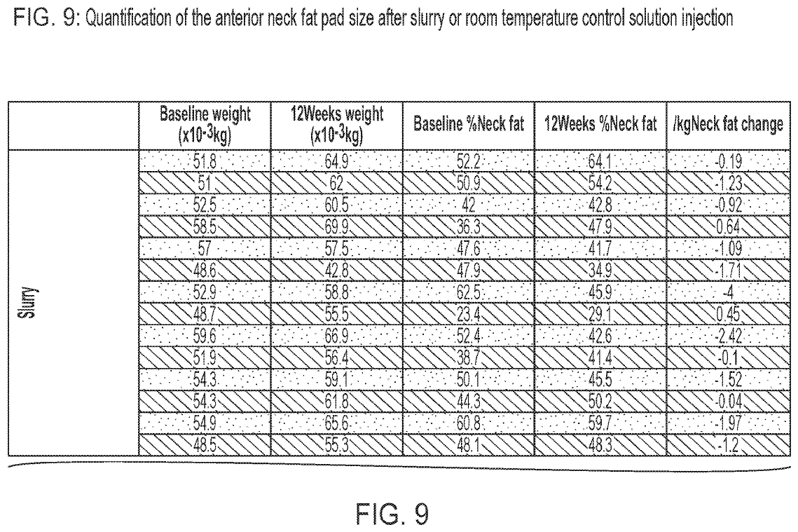

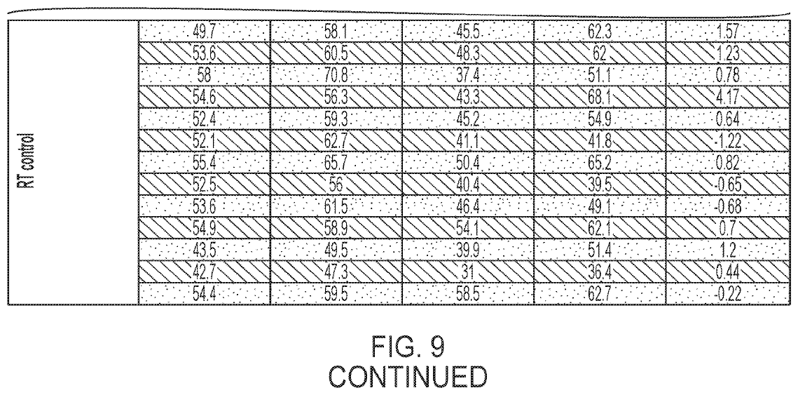

Experiments in mice demonstrate that injection of ice slurry significantly reduces fat from the anterior neck as shown by histological images of neck fat pads and adjacent salivary glands (FIG. 7) and Axial MRI imaging of the anterior neck (FIG. 8, 9) in the slurry-injected group compared with the control group of animals. This experiment demonstrated the reduction in anterior neck fat volume after ice slurry administration independent of total body weight changes.

Twenty weeks old male NZO mice were housed at the Massachusetts General Hospital animal facility in accordance with regulations. Baseline MRI imaging was performed prior to treatment to obtain fat tissue volume measurements. Sixteen animals in the test group were injected with 1.0 ml of cold (−3° C. to −4.8° C.) ice slurry, composed of normal saline (0.9% NaCl)+10% glycerol, into anterior neck fat pad. Slurry was made with a sterilized blender (HGB150, Waring Commercial, Torrington, CT) while cooling the solution below its freezing point, and injected through a 15-gauge needle. In addition to the injection of slurry, mice in the treatment group were subjected to topical cooling. The anterior neck was placed on a bed of ice slurry for 10 minutes prior to the injection, and again 10 minutes immediately after injecting the slurry. Fourteen animals in the control group were injected with the same volume and solution composition, at room temperature. The same treatment procedures were repeated 8 weeks later, and MRI imaging was obtained at the end of the study, 12 weeks later.

Mice were imaged on a Bruker Pharmascan 4.7 Tesla MRI. Axial and coronal Rare T1 (Rare Factor: 4, Tr: 900 ms Te: 13.59 ms, Matrix: 256×256×16 with 0.156×0.156×0.5 mm voxels) and axial Dixon method (Fa: 80 deg, Tr: 4.0/4.8 ms Te: 500 ms with same geometry) sequences were performed at baseline pre-treatment, and at 1-month post second treatment. Time point T1 images were registered using a region of interest difference similarity measure based upon binary images of the brain sections. Fat was isolated using the Dixon images which were also registered using the affine transform parameters obtained from the T1 images. The anterior neck 3D regions of interest for volumetric analysis were manually drawn by blinded observers in front of and lateral to the salivary gland using skull structures as landmarks, and the isolated fat tissue obtained from the Dixon images was quantified from that region. Blinded observers performed image analysis using Amira (Thermo Scientific™) and Matlab (The Mathworks) software. Tissue biopsy samples from the treated neck area were obtained at the end of the study for histologic analysis.

Statistical analysis was conducted using Prism 8 (GraphPad Software, Inc., La Jolla, CA). Paired two-tailed Student's t test was used to compare body weight, and fractional neck fat of animals in each group before and after treatment. Multiple Student's t test with adjusted p value was used to compare body weight between test and control groups before and after treatment. Measured fractional anterior neck fat pad volume in each animal was divided by its body weight at the time of MRI scan (supplementary table 1). Two-tailed Student's t test was used to compare changes in fractional anterior neck fat pad volume from baseline per body weight between treatment group and control group. P value <0.05 is considered significant. All the bar graphs show mean+SEM.

As shown in FIGS. 7A-B and 8A-C, histological analysis of samples harvested 12 weeks after treatment showed no scarring, ulceration or nonspecific damage to the tissue surrounding the treatment site, which included salivary tissues (FIG. 6A). Body weight in both the control and test mice groups at 12 weeks increased significantly in comparison to the baseline (control group 52.11×10−3 kg±1.23×10−3 kg vs 58.93×10−3 kg±1.70×10−3 kg at 12 weeks, p<0.01 by paired two-tailed Student's t test; test group 53.18×10−3 kg±0.94×10−3 kg vs 59.79×10−3 kg±1.77×10−3 kg at 12 weeks, p<0.01) (FIG. 6B and FIG. 8). There was no significant difference in body weight between treatment and control groups at baseline or at 12 weeks.

As shown in FIGS. 7 C, E using MRI imaging, the control mice showed significant increase in the anterior neck fat pads volume per neck tissue volume in the region of interest at 12 weeks post first treatment in comparison with baseline (44.73%±2.00% at baseline vs 54.35%±2.89% at 12 weeks; p<0.01 by paired two-tailed Student's t test) (FIGS. 6C and D and FIG. 8). In contrast, mice in the treatment group did not show any increase in neck fat pad volume at 12 weeks in comparison to the baseline (46.94%±2.66% at baseline vs 46.31%±2.43% at 12 weeks; p=0.78 by paired two-tailed Student's t test) (FIGS. 6C and D and FIG. 8). The difference in fractional anterior neck fat pad volume change from baseline per body weight between treatment group and control group was significant (−1.09/kg±0.33/kg vs 0.68/kg±0.37/kg; p<0.01 by two-tailed Student's t test) (FIG. 6 E and FIG. 8).

This data support that injection of ice slurries can be used as a novel and minimally invasive method for reducing excess upper airway adipose tissue and increasing collagen production without any damage to important surrounding tissue. This procedure can be done quickly in minutes, and easily by inserting a needle through an anterior transcervical approach.

What is claimed is:

1. A method of treating obstructive sleep apnea by inducing new collagen formation in the tongue, the method comprising:
fabricating a sterile ice slurry including water and ice particles;
cooling the sterile ice slurry to a predetermined temperature; and
injecting the sterile ice slurry repetitively into a desired tissue region,
wherein the desired tissue region includes the tongue.

2. The method of claim 1, wherein the predetermined temperature is between minus 10 degrees celcius and 0 degrees celcius.

3. The method of claim 1, wherein the number of ice slurry injections is between 1 and 6 injections.

4. The method of claim 3, wherein the volume of ice slurry for each injection is between 5 ml and 50 ml.

5. The method of claim 1, wherein the sterile ice slurry further comprises a biocompatible surfactant.

6. The method of claim 5, wherein the biocompatible surfactant is glycerol.

7. The method of claim 1, wherein injecting the sterile ice slurry into the desired tissue region comprises:
delivering, via a pump, the sterile ice slurry to the desired tissue region; and
suctioning melted slurry from the desired tissue region.

8. The method of claim 1, wherein injecting the sterile ice slurry into the desired tissue region comprises:
delivering, via a syringe, the sterile ice slurry to the desired tissue region; and
suctioning melted slurry from the desired tissue region.

9. The method of claim 1, wherein injecting the sterile ice slurry into the desired tissue region comprises:
inserting a catheter having an injection port and a suction port into the desired tissue region;
delivering the sterile ice slurry to the desired tissue region through the injection port;
and suctioning melted ice slurry from the desired tissue region through the suction port.

10. The method of claim 1, wherein injecting the sterile ice slurry into the desired tissue region comprises:
inserting a balloon-based catheter into or around the desired tissue region; and
recirculating the sterile ice slurry into the balloon-based catheter.

11. The method of claim 1, wherein the ice particles in the sterile ice slurry have a largest cross-sectional diameter of less than 2 millimeters.

12. The method of claim 1, wherein the tongue is cooled to below 0 degrees celcius following injection of the sterile ice slurry.

13. The method of claim 12, wherein the duration of below zero tongue temperature is at least 5 minutes.

14. The method of claim 3, wherein each re-injection occurs when the tissue temperature rises back to 0 degrees celcius.

15. The method of claim 1, wherein the injection of the sterile ice slurry induces new collagen formation in the tongue.

16. The method of claim 1, wherein the injection of the sterile ice slurry stiffens the tongue.

17. A method for treating obstructive sleep apnea by selectively reducing upper airway adipose tissue, the method comprising:
fabricating a sterile ice slurry including water, ice particles, and glycerol;
cooling the sterile ice slurry to a predetermined temperature; and
injecting the sterile ice slurry into a desired tissue region,
wherein the desired tissue region includes upper airway adipose tissue.

18. The method of claim 17, wherein the upper airway adipose tissue is anterior neck fat pad.

19. The method of claim 17, wherein the upper airway adipose tissue is tongue fat.

20. The method of claim 19, wherein the tongue fat is base of tongue adipose tissue.

21. The method of claim 17, wherein the predetermined temperature is between minus 10 degrees celcius and 0 degrees celcius.

22. The method of claim 17, wherein the sterile ice slurry further comprises a biocompatible surfactant.

23. The method of claim 22, wherein the biocompatible surfactant is glycerol.

24. The method of claim 17, wherein injecting the sterile ice slurry into the desired tissue region comprises:
delivering, via a pump, the sterile ice slurry to the desired tissue region; and
suctioning melted slurry from the desired tissue region.

25. The method of claim 17, wherein injecting the sterile ice slurry into the desired tissue region comprises:
delivering, via a syringe, the sterile ice slurry to the desired tissue region; and
suctioning melted slurry from the desired tissue region.

26. The method of claim 17, wherein injecting the sterile ice slurry into the desired tissue region comprises:
inserting a catheter having an injection port and a suction port into the desired tissue region;
delivering the sterile ice slurry to the desired tissue region through the injection port;
and suctioning melted ice slurry from the desired tissue region through the suction port.

27. The method of claim 17, wherein injecting the sterile ice slurry into the desired tissue region comprises:
inserting a balloon-based catheter into or around the desired tissue region; and
recirculating the sterile ice slurry into the balloon-based catheter.

28. The method of claim 17, wherein the ice particles in the sterile ice slurry have a largest cross-sectional diameter of less than 2 millimeters.

29. The method of claim 17, wherein the anterior neck fat pad is cooled following injection of the sterile ice slurry.

30. The method of claim 17, wherein the tongue is cooled following injection of the sterile ice slurry.

31. The method of claim 17, wherein the injection of the sterile ice slurry reduces the volume of the anterior neck fat pad.

32. The method of claim 17, wherein the injection of the sterile ice slurry reduces the volume of the tongue fat.

33. The method of claim 29, wherein the temperature of the anterior neck fat pad is reduced to a temperature between about minus 10° C. and about 0° C.

34. The method of claim 30, wherein the temperature of the tongue fat is reduced to a temperature between about minus 10° C. and about 0° C.

35. The method of claim 17, wherein injecting the sterile ice slurry into the desired tissue region comprises injecting the sterile ice slurry directly into the tongue or neck fat pad tissue.

36. A method of treating obstructive sleep apnea by inducing new collagen formation in the tongue, the method comprising:
fabricating a sterile ice slurry including water and ice particles;
cooling the sterile ice slurry to a predetermined temperature; and
injecting the sterile ice slurry repetitively into a desired tissue region,
wherein the desired tissue region includes upper airway.
